(19) United States
(12) Patent Application Publication
(10) Pub. No.: US 2005/0187464 A1
(43) Pub. Date: Aug. 25, 2005

(54) Respiratory referenced imaging

(76) Inventors: Vincent B. Ho, N. Bethesda, MD (US); John T. O'Neill, Damascus, MD (US)

Correspondence Address: James Remenick;Powell Goldstein LLP, Intellectual Property Group, 901 New York Avenue, N.W., Third Floor, Washington, DC, 20001-4413, US

(21) Appl. No.: 10/989,111

(22) Filed: Nov. 16, 2004

(30) Foreign Application Priority Data
May 16, 2003 (WO) PCT/US03/15422

(51) Int. Cl. A61B 5/05

(52) U.S. Cl. 600/428

(57) ABSTRACT

Methods, systems and devices are presented that provide improved medical diagnostic and intervention procedures such as magnetic resonance imaging, cardiac imaging, cardiac nuclear scintigraphy, computed tomography, echocardiography, imaging to direct laser ablation, imaging to direct radio frequency radiation ablation, imaging to direct gamma knife radiation therapy, and imaging to direct radiation therapy by respiratory gating. In a preferred embodiment, one or more balloon pressure probes within a catheter are placed into the esophagus and detect pressure within the esophagus to infer respiratory air-flow. Other probes such as those based on fiber optics and other useful materials are described. Many of these devices interact poorly or not at all with magnetic and electromagnetic fields, and are particularly useful for use in respiratory gating of MRI.

### REFERENCE TO RELATED APPLICATIONS

This application claims priority to U.S. Provisional Application No. 60/380,826, entitled “Respiratory Referenced Imaging, Therapy and Intervention,” filed 17 May 2002, which is completely and entirely incorporated herein by reference.

### RIGHTS IN THE INVENTION

This invention was made, in part, with support from the United States Government and the United States Government may have certain rights in this application.

### BACKGROUND OF THE INVENTION

1. Field of the Invention

The invention relates generally to medical diagnostics, medical imaging and more particularly to correction techniques for enhancing the use of imaging in diagnostics, therapy and intervention.

2. Description of the Background

Medical imaging technology and techniques that utilize this technology such as magnetic resonance imaging (“MRI”), computerized tomography, ultrasound, laser ablation therapy, and radiation therapy are becoming more important for diagnosis and therapy as medical science advances. However, the full power of many such techniques is limited by body movement during imaging. This movement often causes spatial mis-registration of signal data and significant blurring of tissue structures on the resultant images. The mis-registration and blurred images are relied on for medical procedures, resulting in less precise diagnostic results and therapeutic intervention.

Motion particularly can affect imaging of inherently mobile structures such as the heart [1-3] and upper abdominal viscera [4]. Two principal forms of physiologic motion are cardiac and respiratory movements. Synchronization of data acquisition with the cardiac cycle via electrocardiogram (ECG) gating for example can minimize cardiac motion blurring [1-3] due to these movements.

Respiratory motion can be minimized by breath hold acquisition or some form of respiratory-gated image acquisition during free breathing [5-15]. Breath holding can reduce respiratory contributions to image blurring and treatment imprecision, which inherently limits spatial resolution. Moreover, involuntary diaphragm motion can occur during a breath hold, which may cause image blurring despite adequate voluntary breath holding as shown by Holland et al. [16]. Furthermore, there can be significant differences in cardiopulmonary measurements such as stroke volume during a breath hold acquisition [17]. Still further, free breathing acquisitions (i.e. tidal respiration) remove temporal limitations that breath holding impose on scanning, and allows improved spatial resolution. Free breathing is highly desired as it is better tolerated by elderly patients [18], which is the target population for many imaging measurements.

Free breathing techniques, however, require a good respiratory trigger to synchronize image acquisition. End-expiration typically is utilized because its duration is relatively longer and because reproducibility of static anatomic position is more reliable during tidal respiration. The earliest form of respiratory-gated image acquisition used a simple elastic strap that is wrapped around the upper abdomen of the patient [5-7]. This technique, called respiratory bellows, monitors a subject's abdominal girth. Increased girth signals inspiration onset and decreased girth signals expiration onset. Early imaging successfully implemented this scheme. However, abdominal distension has not been shown to be a reliable trigger for synchronization of image acquisition in many persons, especially when imaging small structures such as the coronary arteries.

A second form of respiratory gating during tidal respiration employs a quick navigator echo [8, 11-15]. The navigator echo technique uses a fast two-dimensional scan, typically using two orthogonal pulses, and can monitor the relative position of an internal structure. Although any number of intrathoracic structures that include the cardiac silhouette can be used to track intrathoracic respiratory position, the right hemi-diaphragm is typically used for coronary imaging, as the navigator pulses distort the images produced. The navigator echo technique provides a two-dimensional (2D) trigger for respiration. As described above using the right hemi-diaphragm, information from a navigator echo typically is for the superior-to-inferior displacement of the right hemi-diaphragm. Navigator echoes are limited by “diaphragmatic drift” that can occur during prolonged periods of tidal respiration and the inability to place the navigator pulses too close to the region of interest because of image distortion. Diaphragmatic drift results from deviation of the superior-to-inferior diaphragm position over time and out of the “trigger” threshold. This in turn can cause unsuccessful image acquisition.

Despite these needs, the known respiratory compensation methods such as breath holding, chest expansion monitoring, and internal body structure monitoring are fairly rudimentary and generally give poor results. On the other hand, magnetic resonance and other diagnostic procedures are becoming more sophisticated. Accordingly, such limitations become more important and ever more precise compensation schemes are needed.

Thus, improved methods are needed for accurate detection of respiratory phase to ensure proper synchronization of image data from a specific respiratory phase (i.e. end-expiration). Improved methods also would be useful for proper synchronization of inspiratory and expiratory dynamic multiphase imaging. Such information would be useful for imaging cardiovascular blood flow during tidal respiration or for the assessment of respiration itself. Pulmonary MRI is also becoming popular with the introduction of hyperpolarized gases [19-22], but such techniques are limited by body movement. Accordingly, the ability to image the lungs dynamically or to properly synchronize image data during tidal respiration could greatly improve this and other new and to be discovered techniques as well.

### SUMMARY OF THE INVENTION

The present invention overcomes the problems and disadvantages associated with current strategies and designs and provides new devices and techniques for more precise determination of respiratory phase for a wide range of medical technologies including, but not limited to, in particular, magnetic resonance imaging, cardiac imaging, cardiac nuclear scintigraphy, computed tomography, echocardiography, imaging to direct laser ablation, imaging to direct radio frequency radiation ablation, imaging to direct gamma knife radiation therapy, and imaging to direct radiation therapy.

One embodiment of the invention is directed to systems for gating the medical imaging of a patient comprising a device with at least one sensor that is inserted into a body cavity of a patient or that is held over the face of the patient and that generates a respiratory volumetric signal from the detection of at least pressure, temperature, or air flow; and a monitor that accepts sensor information from the device and generates a gating signal for the medical procedure. Another embodiment provides a system for gating the medical imaging of a patient comprising an esophageal catheter having a proximal end and a distal end, with at least one pressure sensor at the distal end, and a monitor at the proximal end that accepts sensor information from the catheter and that generates a volumetric respiratory signal suitable for gating the medical procedure. Yet another embodiment provides a system for gating the medical imaging of a patient comprising, a breathing apparatus having at least one sensor selected from the group consisting of lung pressure sensor, a lung air volume sensor, and an air flow rate sensor and a monitor that accepts sensor information from the apparatus, collects the information over a time period suitable for determining breath inflow and outflow, and that generates a triggering signal suitable for gating the medical procedure. Yet another embodiment provides a system for gating the medical imaging of a patient comprising at least one temperature sensor that is capable of being placed at least orally, nasally or in a space above the mouth in the patient and a monitor that accepts information from the temperature sensor, collects the information over a time period suitable for determining breath inflow and outflow, and generates a signal suitable for gating the medical procedure.

Another embodiment of the invention is directed to systems for provide respiration information for triggering medical imaging of a patient. Such systems comprise a computer capable of receiving respiratory volumetric information from the patient in real time and a stored program in the computer, wherein the stored program saves multiple data points of the respiratory information, determines an optimal respiratory pattern, and analyses the pattern to determine at least one time point selected from the group consisting of the start of inspiration, the end of expiration, the end of deep inspiration, and the end of deep expiration.

Another embodiment of the invention is directed to MRI-compatible esophageal sensors for gating respiratory imaging of a patient, comprising a fiber optic, at least one pressure sensor at or near the distal end of the fiber optic, and a detector at the proximal end of the fiber optic, wherein the sensor comprises less than one percent ferromagnetic material by weight and the distal end of the fiber optic is shaped for insertion into the esophagus of the patient.

Another embodiment of the invention is directed to MRI-compatible esophageal sensors for gating respiratory imaging of a patient, comprising at least one elongated hollow body having a distal end and a proximal end, at least one balloon at or near the distal end of the hollow body and a detector at the proximal end of the hollow body, wherein the sensor comprises less than one percent ferromagnetic material by weight and the distal end of the fiber optic is shaped for insertion into the esophagus of the patient.

Another embodiment of the invention is directed to MRI-compatible esophageal sensors for gating respiratory imaging of a patient, comprising at least one elongated body having a distal end and a proximal end, at least one pressure transducer at or near the distal end of the hollow body that generates an electrical signal and a conductor to transmit a signal from the pressure transducer to the proximal end of the elongated body, wherein the sensor comprises less than one percent ferromagnetic material by weight and the distal end of the fiber optic is shaped for insertion into the esophagus of the patient.

Other embodiments and advantages of the invention are set forth, in part, in the following description and, in part, may be obvious from this description, or may be learned from the practice of the invention.

### DESCRIPTION OF THE INVENTION

Conventional MRI image gating methods using respiratory data often are flawed due to reliance on linear measurements. Linear, or partially linear measurements such as expanded chest size only poorly associate with actual respiratory volume. For example, bellows gating with an elastic strap provides measurements that tend to follow changes in girth (a linear measurement/parameter), as the diaphragm moves along the z-axis as well. Navigator tracking, which typically involves placement of a tracker on the right hemi-diaphragm for cardiac imaging (another linear parameter) yields signals that tend to be linear and less volumetric as well. In contrast, true respiratory gating would utilize signals that correspond more closely to actual intrathoracic pressure or volume, which correspond more closely to three-dimensional parameters.

It was surprisingly discovered that various measurement systems, methods and devices could generate higher quality trigger signals and thus correspond more closely to lung volume and/or pressure. Prior art girth measurement signals do not relate well to actual lung volume. In contrast intra esophageal pressure and lung volume are more linearly related. That is, a plot of intra esophageal pressure versus lung volume shows a greater correlation coefficient (R²) as determined by a linear least squares regression analysis than that obtained by regression of a plot of girth measurement versus lung volume. Preferably the linear correlation coefficient (R²) from the esophageal pressure measurement is more than 0.02, 0.05, 0.1 or even 0.2 higher than the same volumetric measurement on the same individual carried out by the girth measurement.

In advantageous embodiments a “respiratory volumetric signal” is generated by one) a lung pressure sensor (sensor placed within a lung); 2) lung air volume sensor; 3) air flow rate sensor; 4) esophageal pressure sensor; 5) temperature sensor within an oral or nasal passage; 6) pressure sensor within an oral or nasal passage; or 7) sensor (temperature, pressure, or flow rate) within a breathing apparatus.

Embodiments of the invention concern devices, systems and methods that generate or utilize one or more respiratory volume signals for more accurate volumetric measurements. A volumetric signal corresponds with thoracic pressure and/or volume more closely than that obtained with bellows gating. Previous triggering techniques such as those involving chest expansion and breath holding are limited due to the more linear nature and, additionally, longer inherent time constants associated with those measurements.

Various embodiments of the invention utilize faster response temperature sensing, pressure sensing, and/or lung air-flow sensing. These less linear systems, materials, and devices match imaging systems, which penetrate the body with an energy field such as magnetic resonance imaging or radiative therapy.

In preferred embodiments, volumetric respiratory information (from one or more non-linear measurement(s)) are used to inform an imaging procedure such as magnetic resonance imaging, cardiac imaging, cardiac nuclear scintigraphy, computed tomography, echocardiography, imaging to direct laser ablation, imaging to direct radio frequency radiation ablation, imaging to direct gamma knife radiation therapy, and imaging to direct radiation therapy. The volumetric information is generated by one or more sensors, which output signals into a monitor such as a computer. The monitor uses the information to gate and/or convert image data for improved resolution and, in some cases, provide additional diagnostic information to the medical practitioner. Representative steps used for these embodiments and materials are discussed.

Generate Volumetric Data

Volumetric data, as the term is used herein, can be obtained by pressure sensors, temperature sensors, and flow sensors when properly placed within or near the respiration pathway, as summarized below. Space limitations prevent an exhaustive listing of all possible sensors and their methods of use. A skilled artisan, however, informed by this disclosure, will readily appreciate further sensors and methods of their use, including sensors that will be discovered and/or commercialized as instrumentation and engineering technology advances.

Esophageal Catheter Sensors According to an advantageous embodiment of the invention, one or more detectors in the esophageal lumen generate volumetric data associated with respiration. In preferred embodiments the detectors are part of a esophageal catheter, as are generally known in the art. For example, U.S. Pat. Nos. 6,148,222; 5,810,741; 6,159,158; 5,348,019; 4,214,593; 6,066,101 and 6,104,941 describe catheters useful for inserting detectors into an air passageway or wall of such passageway. The materials used, and methods of their use as described in these patents are contemplated for embodiments of the invention.

Advantageously the esophageal catheter has a plastic surface and comprises an elongated body that is positioned within the body, with a distal end within the lower half or lower one third of the esophagus. Other body lumen locations, including, for example, the stomach also may be used to generate (relatively non-linear) signals that correspond to lung volume or pressure. Advantageously the catheter has a pressure sensor at the distal tip. The pressure sensor is inserted into the esophagus and registers local pressure. Such pressure sensors are known and have been used to measure the pressure of solid body parts against the catheter, as for example reviewed in U.S. Pat. No. 5,810,741 issued to Essen-Moller on Sep. 22, 1998.

In practice, intra-thoracic pressure changes correspond well with lung volume changes and/or lung pressure changes. Generally, inhalation causes an air pressure drop in the esophagus and trachea, and a pressure increase in the stomach. In some cases one or more of these pressure signals occurs even though significant inspiration and movement of air from the ambient room to the patient's lungs does not necessarily follow due to, for example, pharyngeal obstruction. These events may be detected and used to inform the imaging procedure. In an embodiment a computer records and monitors this data over a time period of least one inspiration cycle, preferably at least two inspiration, three, or even more than five inspiration cycles. Following such an entrainment period wherein a reference or normal cycle is determined, the computer monitors for a beginning or end of a cycle or cycle portion.

The computer also may monitor for deviation from the determined cycle. The deviation may be seen, for example as an anomalous decrease or increase in a measurement such as pressure or volume. This deviation may directly be used to signal the presence of a problem, may be analyzed further or may trigger a medical intervention to correct the anomaly such as pharyngeal obstruction.

The simultaneous use of two or more sensors at different locations is particularly contemplated for providing this kind of information. For example, a pressure sensor in the stomach may respond more strongly to a muscular effort for inspiration, whereas a pressure sensor in the lower esophagus would be more responsive to actual lung pressure. Monitoring signals from the two sensors would reveal the condition of muscular effort and lowered effect on lung volume and allow further details for more accurate triggering and manipulation of image data to correct for body movements. A sensor may be placed in the upper airway such as the mouth and used to generate a reference signal for calibrating or otherwise improving the accuracy of using signals from one or more other sensors such as a sensor in the esophagus or lung. One or more algorithms may be used, as will be readily appreciated by a skilled artisan, to achieve gating and data manipulation of image data to correct for body movements. In advantageous embodiments a pressure sensor at or near (i.e. within 2 inches, and preferably within 0.5 inch) the distal end is placed within the lower half of the esophagus. A second sensor optionally may be used and may be placed for example in the upper half of the esophagus or the stomach.

An effort to exhale causes analogous events, but in the opposite direction in many embodiments. That is, air pressure may increase in the esophagus and trachea, and a drop in the stomach. When no effort to breathe occurs, the air pressure in these areas will tend to remain constant. A large variety of esophageal catheters with pressure sensors are known and useful for these embodiments as, for example, mentioned in U.S. Pat. No. 6,238,349, issued to Hickey on May 29, 2001; U.S. Pat. No. 5,836,895, issued to Ramsey, III on Nov. 17, 1998; U.S. Pat. No. 5,570,671, issued to Hickey on Nov. 5, 1996; U.S. Pat. No. 5,531,687, issued to Snoke et al. on Jul. 2, 1996; U.S. Pat. No. 5,526,820, issued to Khoury on Jun. 18, 1996; U.S. Pat. No. 5,477,860, issued to Essen-Moller on Dec. 26, 1995; U.S. Pat. No. 5,437,636, issued to Snoke et al. on Aug. 1, 1995; U.S. Pat. No. 5,398,692, issued to Hickey on Mar. 21, 1995; U.S. Pat. No. 5,263,485, issued to Hickey on Nov. 23, 1993; U.S. Pat. No. 5,117,828, issued to Metzger et al. on Jun. 2, 1992; U.S. Pat. No. 5,087,246, issued to Smith on Feb. 11, 1992; U.S. Pat. No. 4,930,521, issued to Metzger et al. on Jun. 5, 1990; U.S. Pat. No. 4,841,977, issued to Griffith et al. on Jun. 27, 1989; and U.S. Pat. No. 4,214,593, issued to Imbruce on Jul. 29, 1980.

Common materials and designs may be used for embodiments wherein a small balloon or other distensible surface is affixed to a piece of catheter tubing and wherein the tubing is connected at its opposite end to an exterior pressure transducer as described in U.S. Pat. No. 4,981,470, issued on Jan. 1, 1991 to Bombeck. In another embodiment a pressure transducer is used that alters an optical signal that is transmitted through a fiber optic to a distal location outside the body. Both embodiments are particularly useful in environments where a high magnetic field is employed for imaging.

A particularly desirable embodiment uses a balloon made from the finger of a latex glove that is affixed to the end of a tube as mentioned in U.S. Pat. No. 5,810,741. The balloon is partially inflated. An air pressure monitor at the proximal end of the catheter connected to the balloon indicates respiratory effort. The lumen of the tube that connects the balloon to the proximal end of the catheter may be filled with a gas such as regular air, or nitrogen, or with a fluid such as water, physiological saline, or oil. The proximal end in this embodiment comprises a pressure transducer that senses a pressure change from the gas or fluid, and generates an electrical signal. The signal in many embodiments is input to a computer monitor, which stores information over a time period of at least one expiration or inspiration. The stored information maybe used to determine a pattern for comparing later signals. In an embodiment a real time signal input from a sensor is used to trigger the imaging system.

Fiber Optic Sensors MRI imaging and other imaging systems may be sensitive to the presence of metal, and particularly ferrous or paramagnetic metal in sensors that are placed on or in a patient body. A balloon-based esophageal pressure detector mentioned above is very useful in this context. In another embodiment of the invention a fiber optic sensor that comprises mostly glass is used to transmit a signal from a sensor to a monitor outside a patient body while interacting less with the imaging system. Preferably the fiber optic glass fiber or fiber bundle comprises at least one sensor and is covered with a plastic sheath. The sensor may be a pressure signal and the fiber optic becomes a catheter that is inserted into the esophagus to provide a pressure signal.

A variety of pressure sensors may be built into the fiber optic and are contemplated for embodiments of the invention. Preferably, at least one pressure sensor is located at or near the distal end of the fiber optic (i.e. within 2 inches of the end and preferably within 0.5 inch from the end) and positioned within the lower half of the esophagus. One suitable sensor is a cantilevered shutter system within a circumferential pressure transmitting membrane wherein the shutter excursion into a gap in the optical fiber varies the amount of light transmitted by the fiber as a function of the external pressure, as described in U.S. Pat. No. 4,924,877, issued to Brooks on May 15, 1990. Another suitable sensor includes an elastic sleeve with a diaphragm light reflector portion such as a single crystal silicon body or a highly reflective material such as aluminum, through which hydrostatic pressure is transmitted as a force acting on a light conductor as described in U.S. Pat. No. 5,018,529 issued to Tenerz et al. on May 28, 1991 and U.S. Pat. No. 5,195,375 issued to Tenerz et al. on Mar. 23, 1993. Yet another useful fiber optic sensor is a mirror interferometer based device such as a U-shaped optical fiber embedded in a silicone rubber probe, wherein changes in optical length result in changes of face-independent light intensity that correspond to changes in pressure, as described in U.S. Pat. No. 5,348,019, issued to Sluss Jr., et al. on Sep. 20, 1994.

These fiber optic based sensors and catheters are particularly desirable because they allow pressure signal generation and transmission by light waves in the presence of strong energy fields such as magnetic fields without generally adversely affecting the imaged signal. Of course a fiber optic catheter may comprise more than one sensing segment adjacent to a particular discrete sensing area and further may comprise more than one discrete sensing area on a single catheter. In an embodiment signals from at least two sensors that are positioned at two or more distances from the lungs (for example in the air passageway or in the esophagus) are compared to obtain more accurate volumetric trigger data compared to that achieved with one sensor alone. One embodiment is a software program that: a) generates and inputs time based volumetric signal(s) from at least two sensors; b) compares changes within signals from one sensor to determine a time based change; c) compares changes within the signals from at least one more sensor for a time based change; d) compares the results from steps b) and c); and e) outputs a decision (to be used by another section of software and/or signal to be used by hardware) that indicates inspiration, expiration or other time based volumetric signal.

Airway Sensors An embodiment of the invention generates volumetric signals from one or more pressure, temperature and/or flow detectors that are held within an air passageway such as a nasal passage, mouth, throat or face mask. Without wishing to be bound by any one theory of this embodiment of the invention temperature, pressure and flow measurements associated with respiration are volumetric and correspond more reliably to respiration volume compared to chest expansion measurements and are particularly useful for triggering image acquisition procedures. A wide variety of sensors may be used for these embodiments.

A thermister may be used as a temperature sensor to indicate volume of air per unit time and is useful in embodiments of the invention. Another sensitive technique for detecting temperature change as is exemplified in U.S. Pat. No. 3,996,928, which shows a bridge circuit that contains three fixed resistors and a variable resistance. The variable resistance is placed in proximity to a patient's nostril, and the subject's exhaling air-flow periodically cools the variable resistance, unbalancing the bridge which may be connected to a difference amplifier. The output signal from the amplifier relates to the amplitude of the air-flow.

A pressure sensor for detecting air-flow directly may be held within a flow stream, allowing response to local pressure changes, in embodiments of the invention. A large variety of pressure sensors are known, such as semiconductor based, fiber optic based, and balloon based. Preferably a sensor holder is used that may be positioned within the nasal lumen, outside of the nose or mouth, or other suitable place in the respiratory pathway. Most preferably the device positions the detector at least 0.5 mm, 1 mm, 2 mm, 3 mm, 4 mm, or even more than 5 mm away from contact with the interior surface of the respiratory pathway, while allowing respired air to contact the sensor. In an embodiment more than one sensor is used and the signals created by the sensors are compared to correct for vagaries in placement and in movement during use. In one such embodiment a fluid or moisture sensor is additionally used to generate information for calibrating a temperature sensor or correcting for contact of the temperature sensor with moisture.

Breathing mask detectors such as pressure detectors and flow detectors are known in the art and are contemplated for embodiments of the invention. For example, U.S. Pat. No. 6,258,039, issued to Okamoto et al. on Jul. 10, 2001 describes a respiratory gas consumption monitoring device having pressure and temperature sensors, which may be used for embodiments of the invention. U.S. Pat. No. 5,660,171, issued to Kimm et al. on Aug. 26, 1997 describes flow sensors for measuring the rate of gas flow in a flow path communicating with a patient, as well as pressure sensing. Temperature, pressure and flow sensors also may be positioned in the nasal cavity to acquire volumetric information.

Other Sensors A wide variety of sensors may be used in embodiments of the invention. For example a pneumotach may be employed to measure instantaneous airflow as described in U.S. Pat. No. 6,286,508. Other devices for volumetric measurements include various pneumotachs (also termed differential pressure flowmeter), measurement of temperature change of a heated wire cooled by an airflow (hot wire anemometer), measurement of frequency shift of an ultrasonic beam passed through the airstream (ultrasonic Doppler), counting the number of vortices shed as air flows past a strut (vortex shedding), measurement of transmission time of a sound or heat impulse created upstream to a downstream sensor (time of flight device) and counting of revolutions of a vane placed in the respiratory flow path (spinning vane) as described for example in Sullivan et al., Respiratory Care, Vol. 29:7, 736-749 (1984) and as described in U.S. Pat. Nos. 4,047,521; 4,403,514; 5,038,773; 5,088,332; 5,347,843; 5,379,650; 5,535,633 and 6,099,481.

Every sensor that generates a signal that corresponds at least partly to volumetric changes in lung volume, either existing or that will be developed in the future is useful in one or more embodiments of the invention. In a particularly advantageous embodiment the sensor generates a less linear (i.e. more volumetric) signal than does a chest girth sensor. The term “less linear” in this context means that if the sensor output (typically a mechanical attribute such as pressure or an electrical signal) is plotted on the Y axis of an X-Y axis with linear time as the X variable, the plot will be less linear than a girth signal plotted from the same physiological condition using a girth measurement device.

A wide variety of pressure sensors may be used such as a pressure-sensitive capacitor, piezoelectric crystal, piezo-resistive transducer, and a silicon strain gauge. Such sensors are described for example, in U.S. Pat. Nos. 6,120,460; 6,092,530; 6,120,459; 6,176,138; 6,208,900; 6,237,398; 5,899,927; 5,714,680; 5,500,635; 5,452,087; 5,140,990; 5,111,826 and 4,826,616 and may be used in medical procedures. These sensors are particularly advantageous because they generally can generate a volumetric signal corresponding to lung volume or pressure when placed and used appropriately.

Systems for Gating Medical Procedures

An embodiment of the invention is a system that combines a volumetric measuring sensor as, for example described above, with a monitor that receives information from the sensor and analyses the received information to determine a gating time for an imaging procedure. In many cases the system comprises a sensor, a device that holds the sensor at a location within or near a patient body and a monitor circuit and/or software for accepting sensed signals and acting upon them. The sensor(s) may be attached to a esophageal catheter, and where extreme resistance to interference with an energy field such as a magnetic field is desired, both the sensor and the catheter may comprise a fiber optic. Another energy resistant embodiment of the invention is a balloon catheter wherein pressure changes in the balloon are transmitted through a tube filled with gas or fluid to a pressure transducer outside of the body. Many other types of sensors, as reviewed above also may be used. Multiple sensors can provide more detailed information to potentially provide more accurate gating signals.

In yet another embodiment information from one or more of the three physio-techniques is continuously monitored to detect, at an earliest time possible, a medical condition during the MRI or other triggered procedure. In one such embodiment, a patient respiration profile is obtained, whereby inhalation and exhalation times are recorded in computer and anomalous events are compared with previous timing. In another embodiment volume of air inhaled and/or exhaled is compared to a baseline and anomalous events used to alert a medical professional in charge.

Most advantageously, a monitor is positioned outside the body and at some distance to avoid interfering with magnetic energy, electromagnetic energy or particle bombardment used for imaging or therapy. When used with a balloon catheter and a pressure coupling fluid or gas, the monitor typically includes a pressure transducer that contacts directly or indirectly with the gas or fluid. The sensor generates electrical signals in response to pressure changes. When used with other devices such as piezo electric pressure sensors, temperature sensors and flow sensors, typically an electrical signal is conducted from the patient body to the monitor.

The monitor generally modifies one or more signals by buffering (altering the impedance) amplifying the signals and/or filtering to remove noise. In many embodiments the signals are stored in computer memory or other memory and then reviewed to find a pattern. In some embodiments the signals are evaluated in real time for specific characteristics and used directly for triggering. Accordingly, the monitor could be as simple as a buffer and threshold signal detector or as complicated as one or more computers that generate and store standard curves and use algorithms to evaluate incoming data. In each instance the monitor generates a “gating signal” that indicates respiration, such as a beginning point of respiration, an end point, or some other repeated feature of the respiration cycle. The gating signal may be a discrete output electrical signal, optical signal, or magnetic signal, a decision point in a computer program or electrical circuit, or one or more mathematical values expressed within or by a computer or by an electrical circuit.

In an embodiment a software program is stored within a computer that physically is part of the monitor or that is attached to it. The software program stores sequential signals from a volumetric sensor that are associated with respiration (lung volume and/or pressure). In an embodiment the program in a first step creates an individualized (normal) profile for a respiration cycle (a completer exhalation, inhalation or combined inhalation/exhalation). In a second step the program compares features of the profile with known or expected features to determine (calculate or select) a type of sensor signal change that indicates the beginning or end of a respiration cycle. In a third step the program monitors sensor data while the data comes in and looks for the determined change. The computer decides when the change is found and triggers another part of the program, another computer or some other output device to gate or control the imagine procedure.

In an embodiment, two or more respiratory profile characteristics, at least one of which is a volumetric measurement as defined herein, are monitored and compared. Possible sampled respiratory characteristics are respiratory flow rate, respiratory pressure, esophageal pressure, stomach pressure, partial pressure of at least one constituent of a patient's respiration and temperature of exhaled air. Calculations of one or more parameters may be carried out as, for example described in U.S. Pat. No. 6,099,481.

A variety of medical procedures utilize imaging and can benefit from embodiments of the invention, including diagnostic procedures such as MRI and CAT, and therapies. Such therapies include, for example, super conducting open configurations for image guided therapy as described by Schenck et al. [23], tumor ablation as described by Cline et al. [24], microwave thermal ablation as described by Chen et al. [25], and radio frequency endocardial ablation using real time three dimensional magnetic navigation as described by Shpun et al. [26]. Results of such therapies may be monitored by, for example, MRI to determine anatomic changes and even temperature changes from the therapy. In each case, proper respiratory gating facilitates improved timing for the therapy either by ensuring proper or improved imaging of, for example, the catheter (i.e. higher detail may be required to see catheter or target structure), potentially augmenting the therapy or simply enabling proper selective timing of ablation.

Magnetic and Radio Field Transparent Materials for Improved Performance

Many of the imaging procedures used in embodiments of the invention utilize strong magnetic (MRI) or radio (x-ray imaging for example) energy fields. These fields penetrate the patient's body and generate an image based on interaction with components of the body. Introduced components such as metals and ceramics used in sensors and leads from sensors to monitors often are MRI sensitive and/or radio opaque. For example, a metal wire used to transmit an electrical signal from a sensor to a monitor circuit may absorb energy from a strong alternating magnetic field and acquire eddy currents big enough to form a spark. Ferrous and other paramagnetic materials in particular cause distortions in the MRI images and should be avoided.

Advantageous embodiments utilize MRI resistant materials and radio transparent materials. Examples of such materials are described in U.S. Pat. Nos. 4,050,453; 4,257,424; 4,370,984; 4,674,511; and 4,685,467, which show forming the conductive element of a monitoring electrode by painting an electrode base with metallic paint or depositing a very thin metallic film on the base, to minimize interaction with the imaging procedure. Another embodiment forms a conductive element such as an electrode lead by applying fine particles of an electrically conductive material, such as carbon, to a base, as described by U.S. Pat. Nos. 4,442,315 and 4,539,995. In yet another embodiment a conductive element is formed from a porous carbonaceous material or graphite sheet, as described in U.S. Pat. Nos. 4,748,993 and 4,800,887. Other MRI compatible materials are described in U.S. Patent No. 60/330,894 entitled “Cardiac Gating System and Method” filed on Nov. 2, 2001 and are particularly desirable for embodiments of the invention that utilize MRI imaging.

These materials also may be used in conjunction with radio imaging techniques. For example, X-ray transmissive materials that comprise electrically conductive carbon filled polymer and/or electrically conductive metal/metal coating on at least a major portion of a side of an electrode may be used as described in U.S. Pat. No. 5,733,324 issued to Ferrari on Mar. 31, 1998. Porous granular or fibrous carbon, optionally impregnated with an electrolytic solution are described in U.S. Pat. No. 4,748,983. Other X-ray transmissive electrical conducting materials that are suitable for embodiments of the invention are described in U.S. Pat. Nos. 4,050,453; 4,257,424; 4,370,984; 4,674,511; 4,685,467; 4,442,315; 4,539,995 and 5,265,679.

Particularly desirable embodiments that are radio transmissive and/or magnetic field transmissive are sensors, masks, sensor holders and catheters that comprise primarily (at least 90% by weight, more advantageous at least 95%, 97%, 98% or even more than 99% by weight) organic polymer such as a medical grade plastic or glass. An esphogeal catheter having a fluid or air filled center with a balloon on the distal end is particularly advantageous as the monitor may be placed outside of the body without contacting the body. Thus, the monitor (pressure transducer, electrical circuits etc.) may contain metal without interfering necessarily with imaging. Another particularly advantageous monitor, which generally has a fast response time is an esophageal catheter comprising an optic fiber with a bend-pressure detector or added pressure detector and which transmits an optical signal outside the body for a distance to connect with a metal containing monitor.

Some piezo electric crystals, particularly those made from polymers are MRI and/or radio energy transparent. Many piezoelectric materials are known that generate electricity in response to pressure and are contemplated such as, for example, discussed in U.S. Pat. No. 4,387,318 issued to Kolm et al.; U.S. Pat. No. 4,404,490 issued to Taylor et al.; U.S. Pat. No. 4,005,319 issued to Nilsson et al. and U.S. Pat. No. 5,494,468 issued to Demarco, Jr. et al. Particularly advantageous are polymers, which can be cast in the form of piezoelectric plastic sheets or other forms. Particularly, polymers known as PVDF polymers are contemplated. The term “PVDF” means poly vinylidene fluoride. The term “PVDF polymer” means either the PVDF polymer by itself and/or various copolymers comprising PVDF and other polymers, e.g., a copolymer referred to as P(VDF-TrFE) and comprising PVDF and PTrFE (poly trifluoroethylene).

PVDF polymers are commercially available as sheets and may be formed to include thin electrodes (to minimize interaction with energy fields) of various metals, e.g., silver, aluminum, copper and tin, as well as known conductive inks or organic polymer (which interact even less) on their opposite major surfaces. The sheets are relatively strong and tear resistant, flexible and chemically inert. Such PVDF polymer piezoelectric materials may be inserted as, for example, long pieces aligned with the long axis of a catheter and positioned in the esophagus. To allow greater flexibility the metal electrode(s) if used may be made from metal(s) of high ductility, e.g., tin and silver, and a known conductive ink including, for example, carbon black or silver particles.

Radio transparent piezo electric sensors are particularly desirable to combine plastic pressure sensors that generate electrical signals with non-metallic conductors. These structures may be electrically isolated from surrounding physiological fluid by a coating, e.g., of polymer such as a silastic polymer, a multiple polymer coat such as silastic polymer on a base of other rigid plastic, or other arrangement, as for example shown in U.S. Pat. No. 6,172,344.

Other embodiments and uses of the invention will be apparent to those skilled in the art from consideration of the specification and practice of the invention disclosed herein. All references and written documents cited herein, for any reason, including all U.S. and foreign patents and patent applications and any priority documents, are specifically and entirely hereby incorporated by reference. It is intended that the specification and examples be considered exemplary only, with the true scope and spirit of the invention indicated by the following claims.

### LITERATURE CITED

1. Hawkes R C, Holland G N, Moore W S, Roebuck E J, Worthington B S. Nuclear magnetic resonance (NMR) tomography of the normal heart J Comput Assist Tomog 1981; 5:605-612.
2. Lanzer P, Botvinick E H, Schiller N B, et al. Cardiac imaging using gated magnetic resonance. Radiology 1984; 150:121-127.
3. Lanzer P, Barta C, Botvinick E H, Wiesendanger H U D, Modin G, Higgins C B. ECG-synchronized cardiac MR imaging: Method and evaluation. Radiology 1985; 155:681-686.
4. Haacke E M, Lenz G W, Nelson A D. Pseudo-gating: Elimination of Periodic motion artifacts in magnetic resonance imaging without gating. Magn Reson Med 1987; 4:162-174.
5. Amoore J N, Ridgeway J P. A system for cardiac and respiratory gating of a magnetic resonance imager. Clin Phys Physiol Meas 1989; 10:283-286.
6. Lenz G W, Haacke E M, White R D. Retrospective cardiac gating: A review of technical aspects and future directions. Magn Reson Imaging 1989; 7:445-455.
7. Chia J M, Fischer S E, Wickline S A, Lorenz C H. Performance of QRS detection for cardiac magnetic resonance imaging with a novel vectorcardiographic triggering method. J Magn Reson Imaging 2000; 12:678-688.
8. Beischer D E, Knepton J C, Jr. Influence of strong magnetic fields on the electrocardiogram of squirrel monkeys (saimiri sciureus). Aerosp Med 1964; 35:939-944.
9. Tenforde T S, Gaffey C T, Moyer B R, Budinger T F. Cardiovascular alterations in Macaca monkeys exposed to stationary magnetic fields: Experimental observations and theoretical analysis. Bioelectromagnetics 1983; 4:1-9.
10. van Genderingen, H. R., Sprenger, M., de Ridder, J. W., and van Rossum, A. C. Carbon Fiber Electrodes and Leads for Electrocardiography during MR Imaging. Radiology 1989; 171: 872.
11. Burch, G. E. History of Precordial Leads in Electrocardiography. Eur. J. of Cardiology 1978; 6: 207-236.
12. Melendiz, L. J., Jones, D. T., and Salcedo, J. R. Usefulness of Three Additional Electrocardiographic Chest Leads (V7, V8 and V9) in the Diagnosis of Acute Myocardial Infarction. Canadian Medical Association Journal 1978; 119: 745-748.
13. Defibrillator/Monitor/Pacemakers. Health Devices 2000; 29:302-334.
14. Schenck J F, Jolensz F A, Roemer P B, et al. Superconducting open-configuration MR imaging system for image-guided therapy. Radiology 1995; 195:805-814.
15. Cline H E, Hynynen K, Watkins et al. Focused US system for MR Imaging-guided tumor ablation. Radiology 1995; 194:731-737.
16. Chen J C, Moriarty J A, Derbyshire J A. Prostate cancer: MR imaging and thermometry during microwave thermal ablation—initial experience.
17. Shpun S, Gepstein L, Hayam G, Ben-Jaim S A. Guidance of radiofrequency endocardial ablation with real-time three-dimensional magnetic navigation system. Circulation 1997; 96:2016-2021.
23. Schenck J F, Jolensz F A, Roemer P B, et al. Superconducting open-configuration MR imaging system for image-guided therapy. Radiology 1995; 195:805-814.
24. Cline H E, Hynynen K, Watkins et al. Focused US system for MR Imaging-guided tumor ablation. Radiology 1995; 194:731-737.
25. Chen J C, Moriarty J A, Derbyshire J A. Prostate cancer: MR imaging and thermometry during microwave thermal ablation—initial experience.
26. Shpun S, Gepstein L, Hayam G, Ben-Jaim S A. Guidance of radiofrequency endocardial ablation with real-time three-dimensional magnetic navigation system. Circulation 1997; 96:2016-2021.

1. A system for gating medical imaging of a patient comprising:
a device with at least one sensor that is inserted into a body cavity of a patient or that is held over the face of the patient and generates a respiratory volumetric signal from detection of at least one of pressure, temperature, or air flow; and
a monitor capable of accepting sensor information from the device and generating a gating signal for medical imaging.

2. A system for gating medical imaging of a patient comprising:
an esophageal catheter having a proximal end and a distal end, with at least one pressure sensor at the distal end; and
a monitor at the proximal end capable of accepting sensor information from the catheter and generating a volumetric respiratory signal suitable for gating medical imaging.

3. A system for gating medical imaging of a patient comprising:
a breathing apparatus having at least one sensor selected from the group consisting of lung pressure sensor, a lung air volume sensor, and an air flow rate sensor; and
a monitor capable of accepting sensor information from the apparatus, collecting sensor information over a time period suitable for determining breath inflow and outflow, and generating a triggering signal suitable for gating medical imaging.

4. A system for gating medical imaging of a patient comprising:
at least one temperature sensor that is capable of being placed at least orally, nasally or in a space above the mouth of the patient; and
a monitor capable of accepting information from the temperature sensor, collecting the information over a time period suitable for determining breath inflow and outflow, and generating a signal suitable for gating medical imaging.

5. The system of claim 1, further comprising an imager capable of receiving and responding to an output signal, wherein the imager is selected from the group consisting of magnetic resonance imaging, cardiac imaging, cardiac nuclear scintigraphy, computed tomography, echocardiography, imaging to direct laser ablation, imaging to direct radio frequency radiation ablation, imaging to direct gamma knife radiation therapy, and imaging to direct radiation therapy.

6. The system of claim 1, wherein the at least one sensor is a pressure sensor selected from the group consisting of a balloon, a piezoelectric transducer and an optical fiber.

7. The system of claim 6, wherein the balloon is connected to the proximal end of the esophageal catheter via a tube that contains a gas or a liquid.

8. The system of claim 1, further comprising electric leads that transmit the sensor information from the device to the receiver.

9. The system of claim 8, wherein the electric leads lack paramagnetic material.

10. The system of claim 8, wherein the electric leads lack materials with significant ferromagnetic properties.

11. The system of claim 8, wherein the electric leads comprise at least 50% carbon.

12. The system of claim 1, further comprising a fiber optic that transmits an optic signal from one or more sensors to the monitor.

13. The system of claim 1, further comprising a fiber optic pressure sensor selected from the group consisting of a cantilevered shutter, diaphragm light reflector, semiconductor light reflector, and mirror interferometry light reflector.

14. The system of claim 1, further comprising at least two sensors positioned at separate locations, wherein signals from the at least two sensors are compared to correct for shifting movements of one or more of the at least two sensors.

15. The system of claim 1, further comprising an elongated portion capable of transmitting a volumetric signal from one or more sensors near or in a patient body to a monitor away from the body, wherein the elongated portion is radiolucent.

16. A medical procedure for a patient selected from the group consisting of magnetic resonance imaging, cardiac imaging, cardiac nuclear scintigraphy, computed tomography, echocardiography, imaging to direct laser ablation, imaging to direct radio frequency radiation ablation, imaging to direct gamma knife radiation therapy, and imaging to direct radiation therapy further comprising:
generating a respiratory volumetric signal from the detection of at least one of pressure, temperature, or air flow from at least one sensor located in or on the patient; and
determining a preselected point on a normal pressure-volume curve for timing image acquisition.

17. A medical procedure for a patient selected from the group consisting of magnetic resonance imaging, cardiac imaging, cardiac nuclear scintigraphy, computed tomography, echocardiography, imaging to direct laser ablation, imaging to direct radio frequency radiation ablation, imaging to direct gamma knife radiation therapy, and imaging to direct radiation therapy further comprising:
generating a respiratory volumetric signal from the detection of at least one of pressure, temperature, or air flow from at least one sensor located in or on the patient; and
determining an optimum respiratory pattern and sample points for image acquisition.

18. The system of claim 1, wherein the signal generated is made within a computer by a stored program.

19. A system for using respiration information for triggering medical imaging of a patient, comprising:
a computer capable of receiving respiratory volumetric information from the patient in real time; and
a stored program in the computer wherein the stored program saves multiple data points of the respiratory information, determines an optimal respiratory pattern, and analyses the pattern to determine at least one time point selected from the group consisting of the start of inspiration, the end of expiration, the end of deep inspiration, and the end of deep expiration.

20. The system of claim 19, wherein the stored program utilizes a normalized pressure volume curve to determine at least one time point.

21. The system of claim 19, further comprising a balloon esophageal catheter that generates respiratory volumetric information.

22. The system of claim 19, further comprising a mouth piece or airway piece that contains at least one sensor for monitoring at least one of temperature, flow rate or pressure.

23. A magnetic resonance imaging-compatible esophageal sensor for gating respiratory imaging of a patient, comprising:
a fiber optic;
at least one pressure sensor at or near the distal end of the fiber optic; and
a detector at the proximal end of the fiber optic wherein the sensor comprises less than one percent ferromagnetic material by weight and the distal end of the fiber optic is shaped for insertion into the esophagus of the patient.

24. The sensor of claim 23, wherein the at least one pressure sensor is selected from the group consisting of a cantilevered shutter, diaphragm light reflector, semiconductor light reflector, and mirror interferometry light reflector.

25. The sensor of claim 23, comprising less than 0.1 percent ferromagnetic material by weight.

26. The sensor of claim 23, which comprises at least two pressure sensors.

27. A magnetic resonance imaging-compatible esophageal sensor for gating respiratory imaging of a patient, comprising:
at least one elongated hollow body having a distal end and a proximal end;
at least one balloon at or near the distal end of the hollow body; and
a detector at the proximal end of the hollow body wherein the sensor comprises less than one percent ferromagnetic material by weight and the distal end of the fiber optic is shaped for insertion into the esophagus of the patient.

28. The sensor of claim 27, which comprises less than 0.1 percent ferromagnetic material by weight.

29. The sensor of claim 27, which comprises at least two balloons and at least two hollow bodies, wherein each balloon is connected to at least one hollow body.

30. A magnetic resonance imaging-compatible esophageal sensor for gating respiratory imaging of a patient, comprising:
at least one elongated body having a distal end and a proximal end;
at least one pressure transducer at or near the distal end of the hollow body capable of generating an electrical signal; and
a conductor to transmit a signal from the pressure transducer to the proximal end of the elongated body wherein the sensor comprises less than one percent ferromagnetic material by weight and the distal end of the fiber optic is shaped for insertion into the esophagus of the patient.

31. The sensor of claim 30, which comprises less than 0.1 percent ferromagnetic material by weight.

32. The sensor of claim 30, wherein the conductor is an organic conductor.

33. The sensor of claim 30, wherein the conductor comprises at least 50% carbon by weight.

34. The sensor of claim 30, wherein the pressure transducer is a piezoelectric crystal.

35. The sensor of claim 34, wherein the piezoelectric crystal comprises an organic polymer.
